(19) United States
(12) Patent Application Publication
(10) Pub. No.: US 2009/0131763 A1
(43) Pub. Date: May 21, 2009

(54) Method for improving in-home patient monitoring

(76) Inventors: Brenton Taylor, Kenwood, CA (US); Geoffrey Deane, Bellevue, WA (US)

Correspondence Address: MARK RODGERS, 1590 SAN ROQUE ROAD, SANTA BARBARA, CA, 93105, US

(21) Appl. No.: 12/218,038

(22) Filed: Jul. 10, 2008

Related U.S. Application Data
(60) Provisional application No. 60/959,691, filed on Jul. 16, 2007.

(51) Int. Cl. A61B 5/00

(52) U.S. Cl. 600/301

(57) ABSTRACT

The invention is a method to improve in-home care for patients who require supplemental oxygen. Built around an intelligent portable oxygen concentrator which can acquire and store patient data and make the data available over the internet, the method allows a physician to access the data over the internet in sufficient detail to justify billing Medicare or other insurance providers, thus incentivising the physician to monitor the patient more often. The data system provider receives a fee for data access thus making the process profitable enough to be sustainable.

### RELATED APPLICATIONS

This application claims priority to U.S. Provisional Application Ser. No. 60/959,691, filed Jul. 16, 2007

### FEDERALLY SPONSORED RESEARCH

Not Applicable

### SEQUENCE LISTING

Not Applicable

### BACKGROUND OF THE INVENTION

The invention relates to in-home medical data collection and reporting, and in particular to in-home situations where an intelligent oxygen concentrator is present. The application is

The application of oxygen concentrators for therapeutic use is known, and many variants of such devices exist. A particularly useful class of oxygen concentrators is designed to be portable, allowing users to move about and to travel for extended periods of time without the need to carry a supply of stored oxygen. Most of these portable concentrators are based on Pressure Swing Adsorption (PSA) or Vacuum Pressure Swing Adsorption (VPSA) designs which feed compressed air to selective adsorption beds. In a typical oxygen concentrator, the beds selectively adsorb nitrogen, resulting in pressurized, oxygen-rich product gas.

The main elements in an oxygen concentrator are shown in FIG. 1. Air is draw in, and typically filtered, at air inlet 1 before being pressurized by compressor 2. The pressurized air is directed by a valve arrangement through adsorbent beds 3. An exemplary adsorbent bed implementation, used in a concentrator design developed by the inventors, is three columns filled with zeolite powder. The pressurized air is directed through these columns in a series of steps which constitute a PSA cycle. Although many different arrangements of beds are possible as well as a variety of different PSA cycles, the result is that nitrogen is removed by the adsorbent material, and the resulting oxygen rich air is routed to a product gas storage device at 4. Some of the oxygen rich air is routed back through the bed to flush out (purge) the trapped nitrogen to an exhaust. Generally multiple beds, or columns in the exemplary device, are used so at least one bed may be used to make product while at least one other is being purged, ensuring a continuous flow of product gas. The purged gas is exhausted from the concentrator at 6.

Such PSA systems are known in the art, and it is appreciated that the gas flow control through the compressor and the beds in a PSA cycle is complex and requires precise timing and control of parameters such as pressure, flow rate, and temperature to attain the desired oxygen concentration in the product gas stream. Accordingly, most modern concentrators also have a programmable controller 2, typically a microprocessor, to monitor and control the details of the PSA cycle and monitor various parameters. Typically, due to the availability of inexpensive processor hardware, the controller can be configured to have significant processing and communications capability in excess of that required to run the concentrator, with no significant cost penalty. Thus the presence of an in-home concentrator provides the possibility of significant functionality which could be applied to patient and caregiver needs.

Patients who require in-home oxygen generally need medical monitoring of other vital parameters, such as blood oxygen saturation, blood pressure, body temperature, body composition and the like. Currently, either the patient must visit a medical facility or be visited in the home by a technician to gather such information. This is both costly and inconvenient. Moreover, under these conditions, the patient monitoring may not happen frequently enough to be effective.

Thus there is clear need for a sustainable process that would provide convenient, frequent, in-home patient monitoring, particularly for patients requiring supplemental oxygen.

Such a process could reduce the overall burden on the healthcare system by alerting clinicians to potential changes in health status before the health of the patient reaches the level where emergency intervention or hospitalization is required to end an acute episode, thus falling within Medicare rules allowing compensation for preventative services.

The key to sustainability of the process is that both physicians and data providers must be compensated for their services. It is the object of this invention to provide a method that results in better, more frequent patient monitoring while providing adequate compensation to all parties and operating within the rules of compensating bodies such as Medicare.

### BRIEF SUMMARY OF THE INVENTION

The invention is a method for improving in-home patient monitoring. The method includes a physician prescribing by an intelligent oxygen concentrator for a patient. This concentrator preferably has the capability to acquire and store patient data and make the data available over the internet or other data transfer system. A data system is created whereby patients are registered with the system and the data acquired by the concentrator may be accessed by the physician on webpages generated by the system. The accessible data is of sufficient detail to justify the physician billing Medicare or other insurance providers for an approved periodic patient check-up. In some embodiments, the data system provider receives a fee from the physician when the patient data is accessed, other payment methods are employed.

In a preferred embodiment, the method includes notifying the physician of events that should trigger access of the data. The notification may be by email or voice message automatically generated by the data system.

In another embodiment, the data system also allows the physician to set limits on portions of the data, and when the system acquires data that is outside these limits, this constitutes an event that triggers physician notification. The time for the next approved check-up may also constitute an event that triggers physician notification.

In other embodiments, the method may also include interfacing patient monitoring devices including at least one of a pulse oximeter, a blood pressure monitor, a temperature monitor, electronic scale, body composition analyzer or a spirometer, to the programmable controller of the intelligent oxygen concentrator whereby data from these devices is also accessible from the data system.

### BRIEF DESCRIPTION OF THE DRAWINGS

The understanding of the following detailed description of the invention will be facilitated by referring to the accompanying figures.

FIG. 1 shows the general elements of gas concentrators as applicable to the invention.

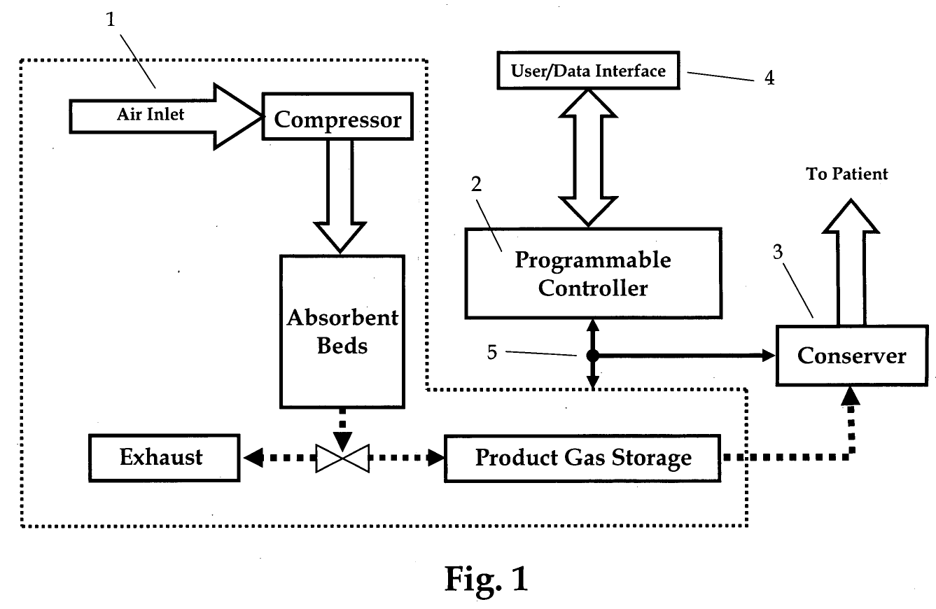

FIG. 2 illustrates the physical set-up that supports the invention.

FIG. 3 is a flow chart of an embodiment of the invention.

### DETAILED DESCRIPTION OF THE INVENTION

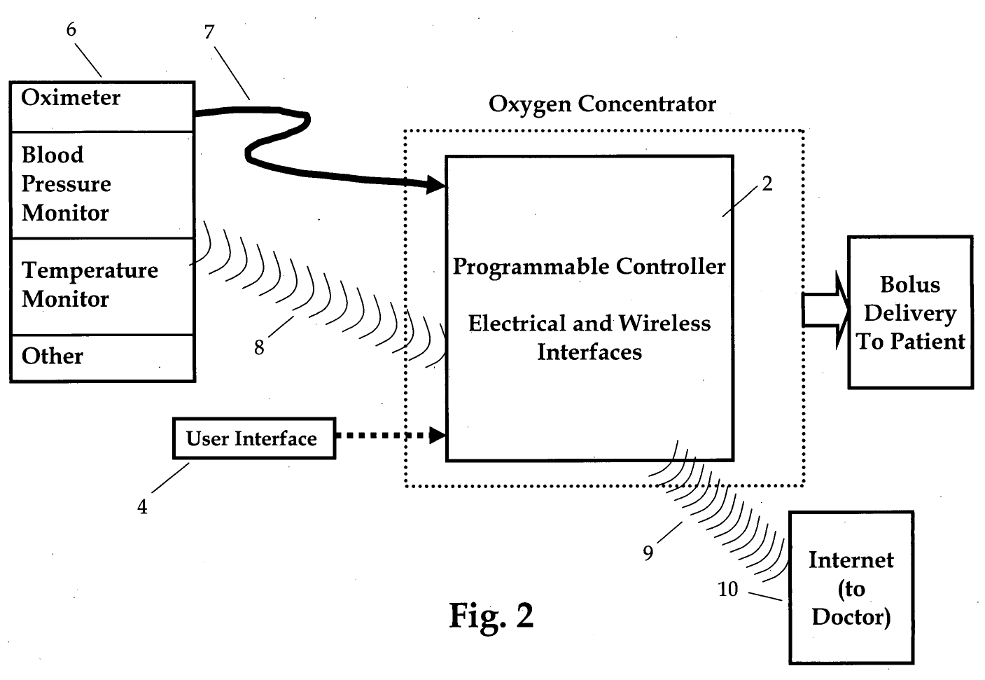

Referring to FIG. 2, the physical set-up required to support the novel method is illustrated. Such a system is described in a co-pending application, docket number 0020701 by the same inventors, and that application is incorporated by reference in its entirety. The central element is the oxygen concentrator, and in particular the programmable controller 2. As described in the co-pending application, such a concentrator is capable on its own, by monitoring various operational parameters, to collect a useful and varied set of patient data. To increase the value and completeness of data collected by the concentrator, the programmable controller may be interfaced to one or more of a suite of medical monitoring devices 6. These devices may include a pulse oximeter, blood pressure monitor, temperature monitor, electronic scale, body composition analyzer, or other more respiratory specific devices, such as spirometers. The interface may be through a wired connection 7, either through general purpose interfaces such as USB or dedicated device-specific interfaces if required. One skilled in the art will readily comprehend a variety of suitable electrical interface panels and the like. Preferably, devices would be used that connect to the controller wirelessly 8 as such an arrangement would be more convenient for the patient. The Bluetooth or Zigbee standard is an example of suitable wireless system for connecting appliances to a controller.

The controller will contain a software application to acquire data from itself and the suite of medical monitors if available. This application preferably also performs other functions. A particularly useful function would be to remind the patient to use the monitors at appropriate times and intervals, possibly using the concentrator's user interface 4 or a dedicated interface system auxiliary to the concentrator.

To support the novel method, the controller will have a communications channel 9 to the outside world, and will use this channel to communicate with the patient's doctor or caregiver. This interface could be a variety of wired or wireless interfaces but must result in a connection to the internet 10. Connection to the Internet facilitates a web-page approach to presenting patient information to the physician. Such an approach is particularly convenient both in terms of flexibility of data management and presentation, as well as providing universal access from a wide variety of locations and connection devices, ie office computers, PDA's, laptops, cell phones etc, allowing for convenient patient monitoring at any time or location.

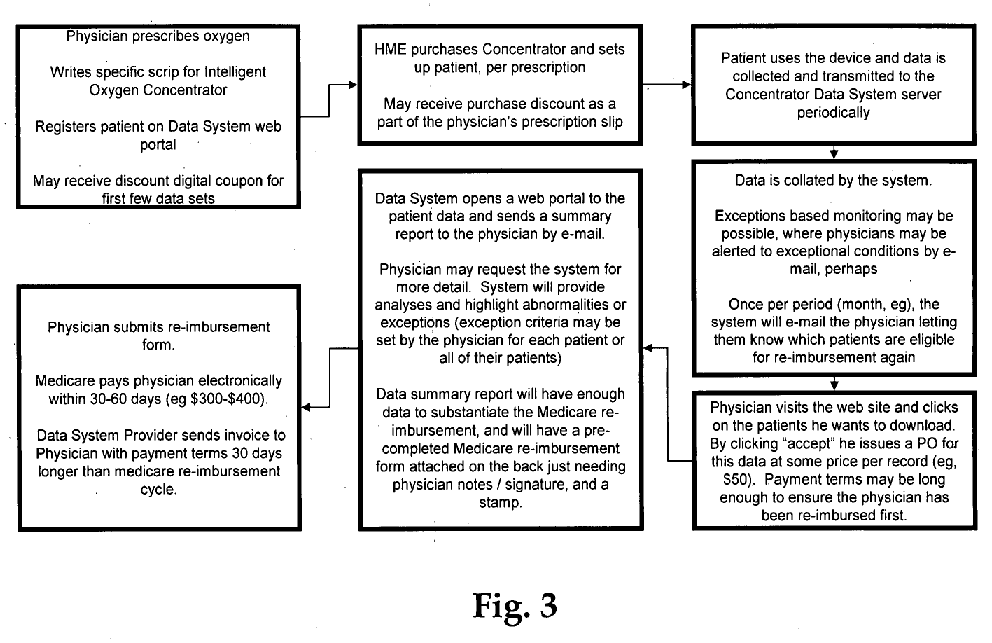

A preferred detailed embodiment of the invention is shown in FIG. 3. Various details of how and when data is collected, bills and fees are invoiced and paid and the like are shown in the Figure. These represent the inventors' preferred implementation, but modifications to many of these details will be readily apparent to practitioners, and these modifications are within the scope of the invention in is broadest sense

The invention ties together the various entities that participate in a home-care scenario involving supplemental oxygen. It does so in a fashion that is sustainable and results in better preventive care for the in-home patient.

The entities are as follows:

1. The insurance organization that ultimately pays for all services and equipment. Medicare is a likely organization to be involved in a supplemental oxygen scenario. Medicare has very strict rules covering what is a billable service and how much compensation is allowed. For purposes of this invention, one service that is allowable is periodic check-ups of a nature that can be shown to help decrease more expensive downstream emergency or hospital care.
2. The HME generally is the actual provider of the equipment itself. The HME must operate in the margin between what it pays for equipment and maintenance and what it can be paid by Medicare.
3. The concentrator manufacturer is generally under intense price pressure from the HME. In order to maintain a supplier base, it would be advantageous for the manufacturer to have sustainable revenue.
4. The patient generally is not highly mobile, and thus it is difficult for the patient to receive enough preventative care since currently such care requires direct contact with a caregiver.
5. The physician is typically in a situation where increased patient contact during the hours available is not possible.

So currently, the patients are not getting enough preventive care, the manufacturers are experiencing difficulty staying in business, the physicians are not receiving perfectly legitimate check-up compensation, and Medicare is burdened with more high-cost hospital care than would be necessary if better preventative measure were taken

The invention as detailed in FIG. 3 addresses many of these issues. Using the concentrator system as described above, the capability of taking and storing in-home medical data and presenting the data on the internet is possible. A data system provider, which preferably is the concentrator manufacturer, builds a system around this data. This system must:

1. Allow for both physician access and input, such as acceptable limits for parts of the data, input either directly by the physician or through documents the physician completes and submits. The documents could also be filled out by the HME, or by the manufacturer.
2. Acquire and organize the data in a web accessible form, in such a fashion as to be acceptable to Medicare or other compensating bodies.
3. Preferably automate, or otherwise make convenient, physician notifications, report generation, and billing.
So in the broadest sense, the invention requires a physician to prescribe an oxygen system with the required capabilities. The data system provider makes the data acquired by the concentrator available to the physician over the Internet, which means the physician is much less time-constrained as to when he can access and analyze the data. The result is the data system provider collects a fee from the physician for data access, the physician collects a larger examination fee from the insurance organization, the patient gets better, more frequent, preventative care resulting in better health and lower costs to the insurance provider, and the HME ends up with patients who remain at home longer before emergency or hospital care is needed. Of course, such a system could also be implemented without fees, as an incentive for improved care, or fees could be paid directly to HME's or providers from Medicare. Also alternative fee arrangements are possible. For instance, the insurance payor may directly compensate the data provider or the equipment provider. For a case where the data and equipment providers are separate entities, one of them may provide the other compensation.

We claim:

1. A method for improving in-home patient monitoring comprising;
prescribing by a physician an intelligent oxygen concentrator for a patient, wherein the concentrator has the capability to acquire and store patient data and make the data available over the internet,
creating a data system whereby patients are registered with the system and the data acquired by the concentrator may be accessed on webpages generated by the system; and,
accessing the patient data over the internet in sufficient detail to justify the physician billing to the insurance payor Medicare for approved periodic patient check-up.

2. The system of claim 1 further comprising notifying the physician of events that trigger access of the data.

3. The method of claim 2 further comprising setting limits on portions of the data by the physician, wherein acquiring data that is outside these limits constitutes an event that triggers physician notification.

4. The method of claim 2 wherein the time for the next approved check-up constitutes an event that triggers physician notification.

5. The method of claim 2 wherein notification is by email automatically generated by the data system.

6. The method of claim 1 further comprising;
interfacing patient monitoring devices including at least one of a pulse oximeter, a blood pressure monitor, a temperature monitor, electronic scale, body composition analyzer or a spirometer, to the programmable controller of a portable oxygen concentrator whereby data from these devices is also accessible from the data system.

7. The system of claim 1 wherein the physician receives remuneration from the payor beyond that normally given for patient check-ups.

8. The system of claim 1 wherein the data system provider receives remuneration from the physician for supplying acquired data.

9. The system of claim 1 wherein the data system provider receives remuneration from the equipment provider for supplying acquired data.

10. The system of claim 1 wherein the data system provider receives remuneration from the payor for supplying acquired data.
